(19) United States
(12) Patent Application Publication
(10) Pub. No.: US 2006/0124442 A1
(43) Pub. Date: Jun. 15, 2006

(54) Device capable of removing contaminants from a fluid

(76) Inventors: Richard S. Valpey III, Lindenhurst, IL (US); Joel E. Adair, Racine, WI (US); Padma Prabodh Varanasi, Racine, WI (US); Matthew A. Jones, Columbus, OH (US); Daniel J. Plankenborn, Milwaukee, WI (US)

Correspondence Address: S.C. JOHNSON & SON, INC., 1525 HOWE STREET, RACINE, WI, 53403-2236, US

(21) Appl. No.: 11/011,862

(22) Filed: Dec. 14, 2004

(51) Int. Cl. C07C 1/00; B01D 47/00; B01D 53/00; C02F 1/78; B01J 19/12; B01D 46/00

(52) U.S. Cl. 204/157.15; 422/186.3; 210/192; 96/223; 96/224; 204/157.3

(57) ABSTRACT

A fluid purifying device having various embodiments, each of which is capable of removing airborne molecules, such as contaminants, malodors, volatile organic compounds (VOCs), pathogens, mold and bacteria, using the combination of visible light and a photocatalyst that has been either treated with a photosensitizer or has been treated in some other manner to lower the energy required to activate the photocatalyst (i.e., by having it generate free radicals) to that emitted by visible light bulbs. Preferably, the device is an air purifying device; however, the device could also be used as a liquid (e.g., water) purifying device.

### FIELD OF THE INVENTION

This invention relates to methods of removing contaminants, such as malodors, volatile organic compounds (VOCs), pathogens, mold and bacteria from a fluid, such as air or water, using a combination of light and a photocatalyst. In particular, this invention relates to fluid purifying devices that incorporate this kind of technology.

### BACKGROUND OF THE INVENTION

In the presence of moisture or oxygen, the combination of ultraviolet light and titanium dioxide yields free radicals. These free radicals remove contaminants from fluids, such as air or water. This technology, which is well known, has been applied to air and water purifying devices.

For example, U.S. Pat. No. 4,892,712 to Robertson, et al. relates to a device that removes, reduces or detoxifies organic pollutants from a fluid, such as air or water, by causing the fluid to contact a matrix made of a substrate onto which a fixed anatase TiO₂ or other photoreactive metal semiconductor material is bonded, in the presence of an ultraviolet wavelength. The substrate has: a large surface area for coating with the photoreactive material; a porous construction such that the fluid to be treated can thoroughly contact the coated surfaces; and sufficient transparency to ultraviolet light to ensure that all the coated surfaces receive such light at an adequate energy level to ensure the catalytic or photoreactive effect. The substrate can be made of a loosely packed mass of woven or spun glass wool, or a length of fiberglass mesh that has been wound into a cylindrical, multi-layer sleeve having a number of convolutions superposed on each other.

However, conventional fluid purifying devices are deficient in various respects. For example, they often contain absorbents, such as activated carbon, which impose back pressure limitations on the devices, causing relatively slow migration of air through the devices for purification. These limitations, in turn, prevent such devices from being able to rapidly remove airborne contaminants from the air. To overcome the back pressure limitations and to allow for more rapid removal of contaminants, relatively large amounts of energy are required to move air through the devices, making them inefficient.

For example, U.S. Pat. No. 5,078,971 to Matuda, et al. and U.S. Pat. No. 4,955,208 to Kawashima, et al., each relates to a deodorizer device for use in a refrigerator that has an absorbent, such as activated carbon, which is capable of absorbing odor components from air, a layer of a photocatalyst deposited on the surface of the absorbent, and an ultraviolet light source for illuminating the photocatalyst layer to excite it. Air containing odor components is forced by a blower to flow through the absorbent.

Another problem with conventional fluid purifying devices is that they are expensive and somewhat dangerous to operate since they utilize ultraviolet light. Also, fluid purifying devices that utilize TiO₂ and that are illuminated with ultraviolet light, generate ozone. High ozone levels damage lung tissue. For example, the devices described in the previously-noted patents require an ultraviolet light source to operate. We are aware of a few patents that suggest the possibility of replacing, in the air purifying system, the ultraviolet light source by a light source in the visible or fluorescent range. However, we do not believe that these patents actually teach a person of ordinary skill in the fluid purifying device art how, without undue experimentation, to practice a method or to make and/or use a device which is capable of removing contaminants from a fluid, particularly air, using visible light (i.e., between about 400 nm and about 700 nm).

For example, U.S. Pat. No. 5,874,701 to Watanabe, et al. relates to a process for photocatalytically treating a hospital room contaminated by bacteria or an interior environment bearing airborne malodorous substances. The process involves depositing a thin film of photocatalyst made of a semiconductor, such as titanium dioxide, on the inner wall of the hospital room or living space. The photocatalytic thin film is irradiated by a light source suitable for general lighting applications, such as a fluorescent lamp, and is photoexcited by the small amount of ultraviolet radiation included in this light. The bacteria and chemical compounds deposited on the photoexcited thin film are photodecomposed. The wattage of the fluorescent lamp, as well as the distance between the photocatalytic thin film and the lamp, are selected such that the intensity of the UV wavelengths, which have a higher energy than the band gap energy of the photocatalyst, is 0.001-1 mW/cm². Consequently, the Watanabe, et al. patent does not describe an air purifying system that operates using a photocatalyst that is, in fact, photoexcited by visible light wavelengths.

Moreover, U.S. Pat. No. 5,790,934 to Say, et al. relates to an apparatus for photocatalytic fluid purification that removes contaminants and microorganisms. The fluid stream to be treated is said to be water or air. Specifically, the Say, et al. patent describes a compact reactor for the photocatalyzed conversion of contaminants in a fluid stream, which includes a photocatalyst disposed on a support structure with a light source in optical proximity to the support structure to activate the photocatalyst. The Say, et al. patent notes that suitable photocatalysts for use in the reactors include semiconductors, such as titanium dioxide. Furthermore, the patent notes that the photocatalyst may be overcoated with a thin film of photo-oxidation-resistant dye (e.g., metal ion complexes) to expand the range of activating wavelengths into the visible range. In addition, the Say, et al. patent suggests that UV lamps or visible light sources can be used, in theory, to activate the reactor, and that the light source should provide a total light intensity at the photocatalyst preferably between 0.1 and 10.0 mW/cm², and most preferably between 0.5 and 2.0 mW/cm². The patent teaches away from using higher light intensities, indicating that using such higher intensities would simply “increase the operating costs of the system while increasing efficiency only marginally.” (See col. 6, lines 25-39). However, for reasons that will be discussed below, we have found that, while the reactor described in the Say, et al. patent might operate successfully to remove contaminants in a fluid stream when exposed to ultraviolet light at the disclosed average activating light intensities, that reactor is not operable in the visible light range at such light intensities.

Finally, U.S. Pat. No. 6,524,447 B1 to Carmignani, et al. relates to an apparatus and a method for the photocatalytic purification and ultrapurification of water. Water containing organic, inorganic and/or biological contaminants is directed through an open cell, three-dimensionally-reticulated, fluid-permeable, semiconductor unit. Within the unit, a semiconductor surface capable of promoting electrons from its valence band to its conduction band, when exposed to a photoactivating light source, removes the contaminants through a photocatalytic reaction. While the focus in the Carmignani, et al. patent is on the use of ultraviolet light as the photoactivating light source in the method and device disclosed therein, the patent does suggest, at column 6, lines 51-60, that the band gap energy of titanium dioxide can be shifted to the visible spectrum (400 nm-700 nm) by coating the surfaces with a transitional metal, such as ruthenium, or combinations of such a metal with other compounds. However, the Carmignani, et al. patent provides no details as to how to render such a visible light system operable.

Similarly, U.S. patent application Publication No. US 2002/0172627 A1 to Aoyagi describes a system for decomposing harmful substances by using a photocatalyst, in which fluid containing such harmful substances enters the system through an inlet and is discharged from an outlet. The system contains a rotator having a photocatalyst fixed on its surface and installed in a flow passage of the fluid in such a manner that it may be rotated. A light source for irradiating light to the photocatalyst (which the Aoyagi patent publication states can be visible light) is also included in the system. The fluid in the system is agitated by rotating the rotator while light is irradiated to the photocatalyst. This increases the frequency of contact between harmful substances and the photdcatalyst, such that the harmful substances are decomposed at a high decomposition rate. However, like the Carmignani, et al. patent, the Aoyagi patent publication provides no details as to how to render the visible light system operable.

### SUMMARY OF THE INVENTION

Accordingly, there is a need for an air purifying device that can safely and effectively remove airborne contaminants using visible light, and at a reasonable cost. We have successfully developed a method and a device for purifying air that is capable of removing contaminants from a fluid, preferably air, by being photoexcited with a visible light source. While our invention is described primarily with reference to the purification of airflows, we believe that the features of our invention may also be effectively applied to the purification of water flows.

Unless otherwise specified, as used herein, the term “light intensity” refers to the approximate light intensity over the entire spectrum of wavelengths of light or “total light intensity.”

In one aspect, our invention provides a fluid purifying device comprising a chamber through which a fluid stream can flow, a photoreactive surface in the chamber, and a visible light source in the chamber. The photoreactive surface comprises a photocatalyst and a photosensitizer. The photosensitizer has a local maximum absorbance in the visible light range at a wavelength (λₘₐₓ), such that the photoreactive surface is activatable by visible light having an intensity greater than about 10 mW/cm² in the wavelength band of λₘₐₓ±12 nm. The visible light source is positioned in the chamber and delivers visible light to the photoreactive surface at an intensity greater than about 10 mW/cm² in the wavelength band of λₘₐₓ±12 nm. The activated photoreactive surface reduces or eliminates contaminants in the fluid stream.

Preferably, the fluid stream comprises either water or air.

The photoreactive surface preferably comprises a photocatalyst and a photosensitizer. Preferably, the photocatalyst is titanium dioxide. Preferably, the photosensitizer is selected from the group consisting of: (1) blackberry extract and (2) a photosensitizer complex of formula (Ia) MX₃Lₜ or (Ib) MXYLₜ, in which: M is a transition metal preferably such as ruthenium and osmium, but also such as iron, rhenium and technetium; each X is a co-ligand that can be, for example, preferably NCS—, but also Cl⁻, Br⁻, I⁻, CN⁻, NCO⁻, H₂O, NCH²⁻ and pyridine unsubstituted or substituted by at least one group selected from vinyl, primary, secondary or tertiary amine, OH and C₁₋₃₀ alkyl; Y is a co-ligand that can be o-phenanthroline, or a 2,2′-bipyridine that is unsubstituted or substituted by at least one C₁₋₃₀ alkyl; and Lₜ is a tridentate ligand containing heterocycles such as pyridine, thiophere, imidazole, pyrazole or triazole, carrying at least one carboxylic, phosphoric or hydoxamic acid or a chelating group.

Even more preferably, the photocatalyst comprises titanium dioxide and the photosensitizer is selected from the group consisting of blackberry extract, cis-bis(isocyanato)(2,2′bipyridyl-4,4′-dicarboxylato) ruthenium (II), Cis-bis(isothiocyanato)bis(2,2′-bipyridyl-4,4′-dicarboxylato)-ruthenium (II), and Tris(isothiocyanato)-ruthenium(II)-2,2′:6,2″-terpyridine-4,4′,4″-tricarboxylic acid.

In another preferred aspect, the photoreactive surface comprises a Ti—O—N constitution in which nitrogen (N) is incorporated in a titanium oxide crystal.

The contaminants in the fluid stream that can be reduced or eliminated by our invention preferably include malodors, volatile organic compounds (VOCs), pathogens, mold and bacteria.

A device according to our invention also preferably includes a fan to direct the flow of the fluid stream through the chamber, and a power source to operate the fan. The power source is preferably selected from the group consisting of an electrical outlet and one or more batteries. The device also preferably comprises an on/off switch to control the operation of the device.

The light source preferably comprises at least one light emitting diode. Preferably the light intensity of the light source is between about 90 mW/cm² and 110 mW/cm². More preferably, the light intensity of the light source is about 100 mW/cm².

A device according to our invention preferably satisfies the requirements of

$$L \leq V ( \frac{{R}^{2}}{k} ) ( \frac{{μ}^{0.26}}{{ρ}^{0.26} {D}^{1.33}} ) ln ( \frac{{c}_{i n}}{{c}_{out}} ) . \qquad ( Equation 2 )$$

Where L is the length of reactor to bring the malodor concentration to zero (cm);

V is the velocity of pollutant stream including air or water (cm/s);

R is the length scale or dimension for the cross section through which air flows (cm);

k is the geometric constant (dimensionless) and is equal to about 0.1;

μ is the viscosity of the pollutant stream whether air or water (g/cm*s);

ρ is density of air or water depending on the system (g/cm³);

D is the diffusion coefficient of malodor in the air or water depending on the system (cm²/s); and

cᵢₙ and cₒᵤₜ are the concentrations of malodor in the incoming and outgoing pollutant streams, respectively, whether it be air or water (g/cm³).

In another aspect, our invention provides a fluid purifying device comprising a chamber through which a fluid stream can flow, a photoreactive surface in the chamber, and a visible light source in the chamber. The photoreactive surface is activated by visible light having an intensity of at least about 90 mW/cm². The visible light source is positioned in the chamber and delivers a light intensity to the photoreactive surface of at least about 90 mW/cm². The activated photoreactive surface reduces or eliminates contaminants in the fluid stream. The device can preferably be operated to purify the air in a room.

Preferably, the fluid stream comprises either water or air.

Preferably, the photoreactive surface comprises a photocatalyst, such as titanium dioxide, and a photosensitizer. The photosensitizer is preferably selected from the group consisting of: (1) blackberry extract and (2) a photosensitizer complex of formula (Ia) MX₃Lₜ or (Ib) MXYLₜ, in which: M is a transition metal preferably such as ruthenium and osmium, but also such as iron, rhenium and technetium; each X is a co-ligand that can be, for example, preferably NCS—, but also Cl⁻, Br⁻, I⁻, CN⁻, NCO⁻, H₂O, NCH²⁻ and pyridine unsubstituted or substituted by at least one group selected from vinyl, primary, secondary or tertiary amine, OH and C₁₋₃₀ alkyl; Y is a co-ligand that can be o-phenanthroline, or a 2,2′-bipyridine that is unsubstituted or substituted by at least one C₁₋₃₀ alkyl; and Lₜ is a tridentate ligand containing heterocycles such as pyridine, thiophere, imidazole, pyrazole or triazole, carrying at least one carboxylic, phosphoric or hydoxamic acid or a chelating group.

More preferably, the photocatalyst comprises titanium dioxide and the photosensitizer is selected from the group consisting of blackberry extract, cis-bis(isocyanato)(2,2′bipyridyl-4,4′-dicarboxylato) ruthenium (II), Cis-bis(isothiocyanato)bis(2,2′-bipyridyl-4,4′-dicarboxylato)-ruthenium (II), and Tris(isothiocyanato)-ruthenium(II)-2,2′:6,2″-terpyridine-4,4′,4″-tricarboxylic acid.

In another preferred aspect, the photoreactive surface comprises a Ti—O—N constitution in which nitrogen (N) is incorporated in a titanium oxide crystal.

Preferably, the contaminants in the fluid stream that are reduced or eliminated include malodors, volatile organic compounds (VOCs), pathogens, mold and bacteria.

In another preferred aspect, the device additionally comprises a fan to direct the flow of the fluid stream through the chamber, and a power source to operate the fan. Preferably, the power source is selected from the group consisting of an electrical outlet and one or more batteries. The device additionally preferably comprises an on/off switch to control the operation of the device.

In another preferred aspect, the light source comprises one or more light emitting diodes. Preferably, the light intensity emitted by the light source is between about 90 mW/cm² and 110 mW/cm², more preferably about 100 mW/cm².

In yet another preferred aspect, the device satisfies the requirements of:

$$L \leq V ( \frac{{R}^{2}}{k} ) ( \frac{{μ}^{0.26}}{{ρ}^{0.26} {D}^{1.33}} ) ln ( \frac{{c}_{i n}}{{c}_{out}} ) . \qquad ( Equation 2 )$$

Wherein the variables are the same as those defined above with respect to equation 2.

In another aspect, our invention provides a fluid purifying device comprising: a chamber through which a fluid stream can flow; a photoreactive surface in the chamber that is activated by visible light and that comprises a Ti—O—N constitution in which nitrogen (N) is incorporated in titanium oxide crystal; and a visible light source in the chamber, wherein the activated photoreactive surface reduces or eliminates contaminants in the fluid stream. Our invention also provides a method of purifying the air in a room using such a device.

Preferably, the fluid stream comprises water or air.

The contaminants in the fluid stream that are reduced or eliminated by our invention preferably include malodors, volatile organic compounds (VOCs), pathogens, mold and bacteria.

A device according to our invention also preferably includes a fan to direct the flow of the fluid stream through the chamber, and a power source to operate the fan. The power source is preferably selected from the group consisting of an electrical outlet and one or more batteries. The device also preferably comprises an on/off switch to control the operation of the device.

The light source preferably comprises at least one light emitting diodes. Preferably the light intensity of the light source is between about 90 mW/cm² and 110 mW/cm². More preferably, the light intensity of the light source is about 100 mW/cm².

A device according to our invention preferably satisfies the requirements of

$$L \leq V ( \frac{{R}^{2}}{k} ) ( \frac{{μ}^{0.26}}{{ρ}^{0.26} {D}^{1.33}} ) ln ( \frac{{c}_{i n}}{{c}_{out}} ) . \qquad ( Equation 2 )$$

Wherein the variables are again the same as those defined above with respect to equation 2.

### BRIEF DESCRIPTION OF THE DRAWINGS

FIG. 1 shows a detailed drawing of a preferred embodiment of our fluid purifying device.

FIG. 2 shows a schematic drawing of a simple cylindrical reactor design for our fluid purifying device.

FIG. 3 shows a schematic drawing of an alternative reactor design for our fluid purifying device containing a packed or fluidized bed of particles.

FIG. 4 shows a schematic drawing of another alternative reactor design for our fluid purifying device containing randomized fibers.

FIG. 5 shows a schematic drawing of yet another alternative reactor design for our fluid purifying device containing axial fibers or thin tubes.

FIG. 6 shows a schematic drawing of still another alternative reactor design for our fluid purifying device containing concentric sleeves.

FIG. 7 shows a schematic drawing of another alternative reactor design for our fluid purifying device containing a spiral sleeve.

FIG. 8A shows a schematic drawing of an alternative reactor design for our fluid purifying device having an annular structure and an axial flow through the device.

FIG. 8B shows a schematic drawing of an alternative reactor design for our fluid purifying device having an annular structure and a rotational flow through the device.

FIG. 9 shows a schematic drawing of another alternative reactor design for our fluid purifying device having an annular structure and a pleated wall.

FIG. 10 shows a schematic drawing of yet another alternative reactor design for our fluid purifying device having an annular structure and radial spikes or fibers.

FIG. 11 shows a schematic drawing of an alternative reactor design for our fluid purifying device having an annular structure and a stator core.

FIG. 12 shows a schematic drawing of an alternative reactor design for our fluid purifying device having a spiral structure.

FIG. 13 shows the disappearance of isopropyl alcohol over time in the presence of light, in an experiment described further in Example 2.

FIG. 14 shows the disappearance of isopropyl alcohol over time in the presence of light, in an experiment described in Example 3.

### DETAILED DESCRIPTION OF THE INVENTION

We have developed a fluid purifying device, in various embodiments, that is capable of removing airborne molecules, such as contaminants, malodors, volatile organic compounds (VOCs), pathogens, mold bacteria, and the like, using the combination of visible light and a photocatalyst that has been either treated with a photosensitizer or has been treated in some other manner to lower the energy required to activate the photocatalyst (i.e., by having it generate free radicals) to that emitted by visible light sources. Preferably, our device is an air purifying device; however, we envision that our device could also be used as a liquid (e.g., water) purifying device.

Our device is an improvement over existing purifying devices for a number of reasons. First, since catalytic amounts of titanium dioxide are employed in our device, absorbents, such as activated carbon, can but need not be used. As a result, our device does not require a high energy input to overcome back pressure limitations caused by absorbents. This makes our device more efficient than conventional devices since less energy is expended by our device in moving air through it. Second, because our device operates in the presence of visible light rather than ultraviolet light,.it is less costly and safer to operate than conventional purifying devices that require an ultraviolet light.

In one embodiment, our invention contemplates a fluid purifying device having a chamber containing a photoreactive component made of a photocatalyst, such as titanium dioxide, coated with a photosensitizer. The addition of a photosensitizer lowers the energy required to form free radicals to that emitted by visible light bulbs. The photoreactive component can be laid down on a surface as discussed in more detail later, to create a photoreactive surface.

While we do not wish to be bound by theory, we believe that free radicals are generated by the photosensitizer and photocatalyst (e.g., titanium dioxide) used in an embodiment of our device, as follows:

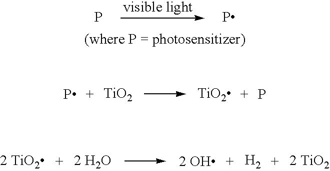

The OH radical (i.e., OH.) is the actual species that destroys contaminants, such as odor molecules. This OH radical only has a survival period of about 30 msec, and so must come in contact with contaminants to be able to destroy them within this limited time period. Moreover, as can be seen from the above series of reactions, the photosensitizer and titanium dioxide are chemically regenerated. Because of this, our fluid purifying device is long-lasting, which is another benefit of our invention.

We have found that when a fluid, such as air, containing unwanted airborne molecules is passed through a chamber containing a photocatalyst, such as titanium dioxide, coated with a photosensitizer, such airborne molecules can be removed in the presence of visible light. Based on our theory of the series of chemical reactions underlying the functionality of our fluid purifying device, we believe that some moisture should be present (and usually is present in any event) in the air to be treated with the device to allow for these reactions to occur. We believe that a fluid, such as water, also could be passed through such a chamber and contaminants could be similarly removed from the water in the presence of visible light. However, we have found that when airflows with high humidity (e.g., above about 5 to 10 parts per million of water) are passed through our device, the efficacy of our device starts to decline somewhat. Accordingly, we would expect to see a similar decline in efficacy when our device is used with water flows. Consequently, when used to treat water flows, the contaminant removal efficacy may be somewhat lower than the experimental values obtained for treatment of airflows.

We envision that various photosensitizers could be used in our device. For example, photosensitizers that might be used in our purifying device are described in U.S. Pat. No. 6,245,988 B1 to Grätzel, et al., the content of which is herein incorporated by reference in its entirety. The Grätzel, et al. patent relates to transition metal complex photosensitizers (i.e., often referred to as “dyestuffs”) and their use in photovoltaic cells containing a nanocrystalline titanium dioxide layer. However, we have found that these photosensitizers also are very effective in our area of interest, namely, fluid purifying devices. More specifically, the Grätzel, et al. patent discloses a photosensitizer complex of formula (Ia) MX₃Lₜ or (Ib) MXYLₜ, in which M is a transition metal preferably such as ruthenium and osmium, but also such as iron, rhenium and technetium; each X is a co-ligand that can be, for example, preferably NCS—, but also Cl⁻, Br⁻, I⁻, CN⁻, NCO⁻, H₂O , NCH⁻² and pyridine unsubstituted or substituted by at least one group selected from vinyl primary, secondary or tertiary amine, OH and C₁₋₃₀ alkyl; Y is a co-ligand that can be o-phenanthroline, or a 2,2′-bipyridine that is unsubstituted or substituted by at least one C₁₋₃₀ alkyl; and Lₜ is a tridentate ligand containing heterocycles such as pyridine, thiophere, imidazole, pyrazole or triazole carrying at least one carboxylic, phosphoric or hydroxamic acid or a chelating group.

Preferably, the following photosensitizers might be used in our purifying device: cis-bis(isocyanato)(2,2′bipyridyl-4,4′dicarboxylato) ruthenium (II) (supplied by Solaronix, Aubonne, Switzerland under the name Ruthenium 505); cis-bis(isothiocyanato)bis(2,2′-bipyridyl-4,4′-dicarboxylato)-ruthenium (II) (also supplied by Solaronix under the name of Ruthenium 535); or tris(isothiocyanato)-ruthenium(II)-2,2′:6′,2″-terpyridine-4,4′,4″-tricarboxylic acid (also supplied by Solaronix under the name of Ruthenium 620, and is also known as “Black Dye”). We also envision that anthocyanins, and particularly cyanins, as discussed in Nerine J. Cherepy, et al., “Ultrafast Electron Injection: Implications for a Photoelectrochemical Cell Utilizing an Anthocyanin Dye-Sensitized TiO₂ Nanocrystalline Electrode”, J. Phys. Chem. B, vol. 101, pp. 9342-9351 (1997), the entire content of which is herein incorporated by reference in its entirety, might be used as photosensitizers in our device. Preferably, blackberry extract, which contains anthocyanins and thus which exhibits photosensitizing properties as discussed in the Cherepy, et al. article, might be used as a photosensitizer in our device.

Suitable photosensitizers also are described in an article by M. K. Nazeeruddin, et al., entitled “Conversion of Light to Electricity by cis-X₂Bis(2,2′-bipyridyl-4,4′-dicarboxylate)ruthenium(II) Charge-Transfer Sensitizers (X=Cl⁻, Br⁻, I⁻, CN⁻, and SCN⁻) on Nanocrystalline TiO₂ Electrodes”, J. Am. Chem. Soc., vol. 115, pp. 6382-6390 (1993), the content of which is incorporated herein by reference in its entirety. The Nazeeruddin, et al. article reports the first systematic study of the luminescence, visible light absorption, electrochemical, and photoredox properties of bis(bipyridyl) Ru(II) complexes having the general formula identified in the title of the article. Photophysical and electrochemical properties of these complexes are listed in Table I. Among these compounds, cis-di(thiocyanato)bis(2,2-bipyridyl-4,4′-dicarboxylate)ruthenium(II) displays outstanding properties as a charge-transfer sensitizer, better than any other known dyestuff to date. Nanocrystalline TiO₂ films coated with this complex harvest light very efficiently. The primary focus of this article is to show that when these films are incorporated in a thin-layer regenerative solar cell equipped with a light-reflecting counter electrode, a solar-to-electric energy conversion efficiency can be attained that is commensurate with that of conventional photovoltaic cells. However, we have found that the photosensitizers described in the Nazeeruddin, et al. article also are suitable for our fluid purifying device.

Conventionally, the intensity of light striking a surface is measured by placing the detector of a radiometer or photometer onto the photoreactive surface, and then by recording the value on the meter. The radiometer or photometer conventionally measures a “total light intensity,” which can be described as the approximate light intensity over the entire spectrum of wavelengths of light. This conventional method tends to be used when one measures the light intensity requirements for a TiO₂ surface, since TiO₂ absorbs a spectral distribution of multiple wavelengths. That is, the conventional method approximates the TiO₂ surface's light requirements.

However, it should be noted that another way to measure the light requirements of a TiO₂ surface when that surface is coated with a photosensitizer is to use a special photometer or radiometer that can actually measure the light intensity at a particular wavelength (or band of wavelengths) of light. This type of measure can be helpful, since light bulbs emit a spectrum of multiple wavelengths, but photosensitizers absorb primarily a narrow band within that spectrum. That is, each photosensitizer has a local maximum absorbance in the visible light range at a predetermined wavelength (λₘₐₓ) Photosensitizers primarily absorb light having wavelengths at or near this wavelength λₘₐₓ. If one uses such a special photometer or radiometer, one can record the spectral distribution of light radiating from a source and then measure the fraction corresponding to the band adsorbed by the photosensitizer. When the measurement is performed in this manner, we surprisingly found that a fluid purifying device will not function in the presence of visible light even when using a photocatalyst, such as titanium dioxide, coated with a photosensitizer, unless the visible light delivered to the photoreactive surface has an intensity greater than about 10 mW/cm² in the wavelength band of λₘₐₓ±12 nm. The width of the band of wavelengths that is absorbed varies somewhat from one photosensitizer to another, but in most instances the majority of the light will be absorbed in the range of λₘₐₓ±12 nm. One suitable light source is a 500 W Xenon arc lamp with a 435 nm Long-pass filter (S244435, Esco Products of Oakridge, N.J.) inserted in the light path to limit the incident light to wavelengths greater than 400 nm, which was used for the experiments herein.

When measured using the conventional total light intensity technique described above, a light source, such as the 500 W Xenon arc lamp with the 435 nm Long-pass filter, typically has a total light intensity of at least about 90 mW/cm². Of course, the total light intensity required to activate the photoreactive surface will depend largely on the overall spectrum of light output by the light source. For the preferred Ru-based photosensitizers noted above, the total light intensity delivered to the photoreactive surface is preferably between about 90 and about 110 mW/cm², more preferably about 100 mW/cm². Preferably, for a photocatalyst (e.g., TiO₂) photosensitized with anthocyanins (e.g., blackberry extract), the total light intensity is greater than about 100 mW/cm². For simplicity, the experiments conducted herein below are described in terms of the total light intensity. Nonetheless, in each of the experiments conducted according to our invention, the light intensity delivered to the photoreactive surface was greater than about 10 mW/cm² in the wavelength band of λₘₐₓ±12 nm.

The light intensity requirement in our fluid purifying device is contrary to the expectation of those having ordinary skill in this art. Such individuals would have expected our light-driven purifying device to operate effectively to remove contaminants from a fluid stream at light intensities of between 0.1 to 10 mW/cm², and more specifically between 0.5 mW/cm² and 2.0 mW/cm², as is the case when ultraviolet light is used. This, for example, is what is taught by U.S. Pat. No. 5,790,934 to Say, et al. considered earlier. Underlying this expectation is an understanding of the incident monochromatic photon-to-current conversion efficiency (IPCE) values for the various types of photosensitizers that might be useful in our inventive fluid purifying device, and particularly the IPCE values for the photosensitizers described in the Nazeeruddin, et al. article, as well as generally in the Grdtzel, et al. patent, as discussed earlier. The IPCE value is defined in the Nazeeruddin et al. article as “the number of electrons generated by light in the external circuit divided by the number of incident photons”, and can be calculated using the following equation provided in the Nazeeruddin, et al. article:

$$IPCE = \frac{[ ( 1.25 \times {10}^{3} ) \times photocurrent density [ μ A / {cm}^{2} ] ]}{[ wavelength [ nm ] \times photon flux [ W / {m}^{2} ] ]} . \qquad ( Equation 1 )$$

The photon flux could also be understood as meaning light intensity. From our perspective, the IPCE value provides a measure of the efficiency by which a photosensitizer is capable of harnessing light energy of a specified wavelength and converting it to electrical energy (as is the interest in the Nazeeruddin, et al. article), or, for our purposes, chemical energy (in the form of free radicals that are capable of decomposing contaminants in a fluid stream).

The authors of the Nazeeruddin et al. article determined IPCE values for the photosensitizers generally described in the title of their paper and found that such photosensitizers had IPCE values exceeding 80% in the visible light range, when the intensity of the incident light was typically in the 0.05 mW/cm² to 1 mW/cm² range. Moreover, FIG. 3 in the Nazeeruddin, et al. article illustrates photocurrent action spectra for nanocrystalline titanium dioxide films coated with bis(2,2′-bipyridyl-4,4′-dicarboxylate)ruthenium(II) complexes (i.e., plotting IPCE % against wavelength/nm). One such photosensitizer (i.e., cis-Di(thiocyanato)-N,N-bis (2,2′-bipyridyl-4,4′-dicarboxylic acid)-ruthenium (II) (i.e., referred to in the graph as RuL₂(NCS)₂) attained an IPCE % plateau as high as 85-90% at such typical light intensities in the visible light range (i.e., at wavelengths between about 500 and about 600 nm). Consequently, given such high IPCE values using such a low intensity light beam, the expectation would be that a similar low intensity light beam would be sufficient to activate the photoreactive surface in our fluid purifying device.

Moreover, a consideration of Equation 1 above for calculating IPCE values reveals that the IPCE value will decrease if the photon flux (or light intensity) is increased. Since it is generally thought to be beneficial to have an IPCE as close to unity as possible, conventional wisdom indicates that increasing the light intensity in a system containing a photocatalyst coated with a photosensitizer dye as discussed earlier not only is not necessary (as suggested by U.S. Pat. No. 5,790,934 to Say, et al.) but also is detrimental to the efficient operation of the system. Consequently, conventional wisdom, as illustrated, for example, in U.S. Pat. No. 5,790,934 to Say, et al. and the Nazeeruddin. et al. article, teaches away from using as high a light intensity as we have found is required to successfully operate our fluid purifying device. Support for our finding that a light intensity greater than about 10 mW/cm² in the wavelength band of λₘₐₓ±12 nm (which, as noted above, corresponds to a total light intensity of at least about 90 mW/cm² when a 500 W Xenon arc lamp with 435 nm Long pass filter is used) is required to operate our fluid purifying device is provided below in Example 3.

In an alternative embodiment of our invention, we envision that our fluid purifying device could incorporate yet other types of photoreactive components that are sensitive to visible light. For example, U.S. patent Publication No. 2002/0169076 A1 to Takeshi, et al., the content of which is incorporated herein by reference in its entirety, discloses photocatalytic materials that have a Ti—O—N constitution in which nitrogen is incorporated in a titanium oxide crystal. These photocatalytic materials exhibit photocatalytic activity in the visible light region. In these materials, nitrogen may be incorporated in the titanium oxide crystal by either one or a combination of processes entailing: substituting a nitrogen atom for an oxygen site of titanium oxide crystal; doping a nitrogen atom within or at an interstitial site of a lattice of a titanium oxide crystal; or doping a nitrogen atom to grain boundaries of titanium oxide. The Takeshi, et al. patent publication also describes preferred manufacturing processes for preparing these photocatalytic materials. Additional information regarding photocatalytic materials that have a Ti—O—N constitution and that we envision might be included in our fluid purifying device can be found, for example, in R. Asahi, et al., “Visible-Light Photocatalysis in Nitrogen-Doped Titanium Oxides”, Science, vol. 293, Jul. 13, 2001, the content of which is herein incorporate by reference in its entirety. See also Shinri Sato, “Photocatalytic Activity of NOₓ-Doped TiO₂ in the Visible Light Region”, Chemical Physics Letters, vol. 123, number 1, 2, Jan. 3, 1986, the content of which also is herein incorporated by reference in its entirety.

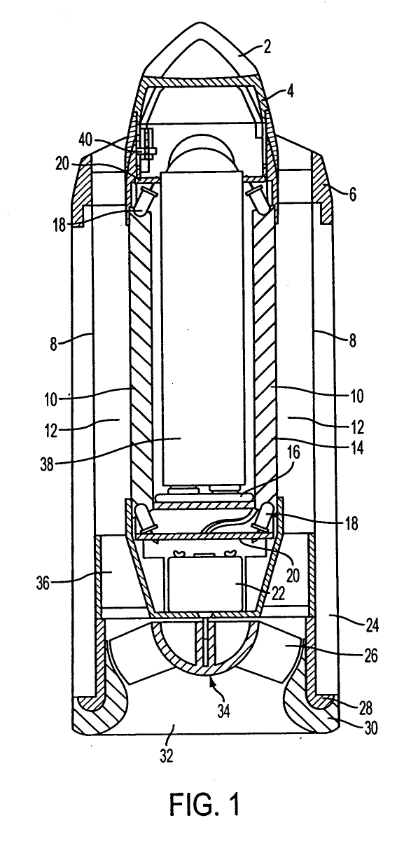

In a particularly preferred embodiment, our fluid purifying device is designed as shown in the side view provided in FIG. 1. As shown in FIG. 1, the device is in the form of a cylinder and treats air by first drawing it in through an inlet nozzle 32. This nozzle's function is to “set up” a certain flow pattern that can best be utilized by the fan 34. The fan's function is to move air efficiently while minimizing turbulence and noise. The jet fan impeller 26 forms part of the fan 34 assembly. The shroud 30 controls the flow of air through the device so that the air must pass through the fan 34. A shroud interlock 28 connects the shroud 30 to the outer sleeve 24 of the device. A motor 22 drives the fan and obtains the necessary electrical energy to do so, for example, from a power source 38 (e.g., such as eight “AA” batteries). An access cap 4 provides access to the power source 38. The electrical energy generated by the power source 38 is transferred to the fan 34 and light-emitting diodes (LEDs) 18 by way of an electrical connection 16. Printed circuit boards 20 are provided at each end of the device by the LEDs 18. Moving air exiting the fan enters a flow-directing stator 36. The purpose of the stator is to introduce a beneficial flow pattern that can best be utilized by the air as it proceeds into the reactor chamber 12. Inside the reactor chamber 12, light from a beam guide 10 excites the photoreactive surface 8. LEDs 18 provide the visible light source that illuminates the beam guide 10 in the device. For example, the device in FIG. 1 might contain eight LEDs, four near the inlet end of the device, and four near the outlet end of the device. The beam guide 10 defines the inner sleeve 14 of the device. The distance between the inside surface of the outer sleeve 24 and the outer surface of the inner sleeve 14 is the distance “R” that would be used in Equation 2 provided and discussed below, whereas the distance between the two sets of LEDs 18 at each end of the device is the distance “L” in Equation 2. Odor molecules and/or other contaminants in the air collide with this surface and are chemically altered. The treated air then exits the device through an exhaust nozzle 2. A top vent diffuser 6 imparts a flow pattern that is beneficial in creating the desired air circulation effect in the environment in which the device is placed. An on/off switch 40 can be used to control the operation of the device.

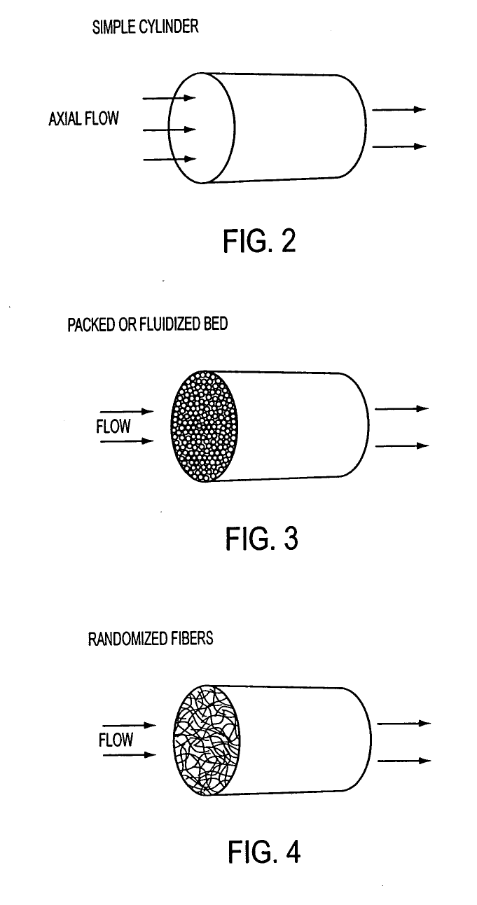

However, our fluid purifying device is not limited only to the reactor design provided in FIG. 1. We envision that our fluid purifying device could take on a number of different designs. Generally speaking, the structure of the photoreactive surface, the positioning of the activating light source, and the type of fluid flow, all can be varied to create numerous designs for our fluid purifying device, as discussed further below. For example, the alternative reactor designs illustrated in FIGS. 2-12, and described further below illustrate how these three variables can be varied to create numerous embodiments of our fluid purifying device. (In this application, including the descriptions of alternative reactor designs provided below, the term “photoreactive component” denotes any photocatalyst that is treated with a photosensitizer or that is altered (e.g., as described in the Takeshi, et al. patent publication), as necessary, to create a “photoreactive surface” that is sensitive to visible light.) While the alternative reactor designs described in FIGS. 2-11 are based on a cylindrical structure, it should be understood that other three-dimensional shapes for the reactor chamber also could be used. Even the coiled or spiraling tube in FIG. 12 could be wound in different ways and yet still be effective.

Various types of photoreactive surfaces might be used in our invention. The key in our invention is to maximize the surface area of any photoreactive surface. We have determined that appropriate dimensions of the photoreactive surface relative to the dimensions of the reactor itself are important to contaminant removal from the fluid passing through the device. Preferably, the reactor should satisfy the following criteria in order to minimize the diffusion time to the surface relative to the residence time of the fluid flow. The characteristic distance over which the fluid (e.g., air or water) flows in the reactor should be equal to or less than the diffusion path length relationship shown in Equation 2.

$$L \leq V ( \frac{{R}^{2}}{k} ) ( \frac{{μ}^{0.26}}{{ρ}^{0.26} {D}^{1.33}} ) ln ( \frac{{c}_{i n}}{{c}_{out}} ) \qquad ( Equation 2 )$$

Where L is the length of reactor to bring the malodor concentration to zero (cm);

V is the velocity of pollutant stream including air or water (cm/s);

R is the length scale or dimension for the cross section through which air flows (cm);

k is the geometric constant (dimensionless) and is equal to about 0.1 for all of the reactor designs disclosed herein (k is determined based on the geometry of the reactor design, and may vary somewhat with changes in reactor design);

μ is the viscosity of the pollutant stream whether air or water (g/cm*s);

ρ is density of air or water depending on the system (g/cm³);

D is the diffusion coefficient of malodor in the air or water depending on the system (cm²/s); and

cᵢₙ and cₒᵤₜ are the concentrations of malodor in the incoming and outgoing pollutant streams, respectively, whether it be air or water (g/cm³).

That is, Equation 2 provides insight as to the maximum distance that a contaminant molecule within the reactor of our fluid purifying device can be expected to successfully diffuse in order to arrive at a photoreactive surface and be broken down, given a particular flow rate of fluid containing contaminant molecules through a reactor of a particular length.

In addition to varying the type of photoreactive surface used in our fluid purifying device, the type of fluid flow also can be varied, as suggested earlier. For example, the fluid flow through a reactor can be axial, namely, a fluid flow that moves directly through the reactor. Alternatively, the fluid flow through the reactor can be rotational so as to create a more turbulent (i.e., convective) flow pattern within the reactor. Such rotational fluid flow can render the fluid purifying device more effective since it tends to increase the number of times that contaminant molecules come in contact with a photoreactive surface, and this, in turn, increases the rate of fluid decontamination by the device. That is, the convective motion of contaminants toward the photoreactive surface enhances the rate at which the series of degradation reactions referred to earlier occurs.

As well, the position of the light source used to activate the photoreactive surface also can be placed in a variety of locations in our fluid purifying device, such as on either end of the device, along the inner edge of the device, or even along the central axis of the device.

For example, a simple structure for our fluid purifying device might take the form of a cylinder, as depicted in FIG. 2, with axial fluid flow through the cylinder to be decontaminated. Although FIG. 2 does not show the location of the activating light source or of the photoreactive surface, the light source might be positioned in any number of locations, including, for example, each end of the cylinder, and the photoreactive surface might be established along the inner walls of the cylinder.

To increase the effectiveness of the fluid purifying device shown in FIG. 2, a number of modifications can be made to the photoreactive surface, as shown in FIGS. 3, 4, 5, 6 and 7.

FIG. 3 shows a cylindrical fluid purifying device that has a reactor chamber containing a large plurality of particles, the surface of each particle being coated with a photoreactive component. It is these coated particles that collectively form the photoreactive surface in this design of our fluid purifying device. An axial fluid flow can be passed through the reactor chamber of this device for purification. The plurality of particles can be fluidized or packed. If fluidized, the particles will float in the stream of fluid passing through the reactor chamber. On the other hand, if the particles are packed, then they will stay fixed in one location while still allowing fluid flow to pass through the reactor chamber. The particles could be made entirely of TiO₂ and coated with photosensitizer or they could be made by coating any durable component, such as glass or plastic with photosensitizer-coated TiO₂. In this embodiment, the activating visible light source could be positioned anywhere that is convenient, including along the internal walls of the reactor chamber.

Alternatively, FIG. 4 shows a reactor for our purifying device that contains a loosely packed column of randomized fibers (e.g., made of glass wool or of polymeric fibers) onto which is deposited a suitable photoreactive component. Therefore, the surface of these randomized fibers becomes the photoreactive surface. FIG. 4 also shows a direct or axial fluid flow passing through the reactor chamber. Although the visible light source required to activate the photocatalytic degradation of contaminants in this reactor could be placed in a number of locations so long as it sufficiently illuminates the glass wool to render the reactor an effective fluid purifying device, preferably, the light source would be placed peripherally, along the inside wall or walls of the reactor. Alternatively, the glass wool itself could be illuminated.

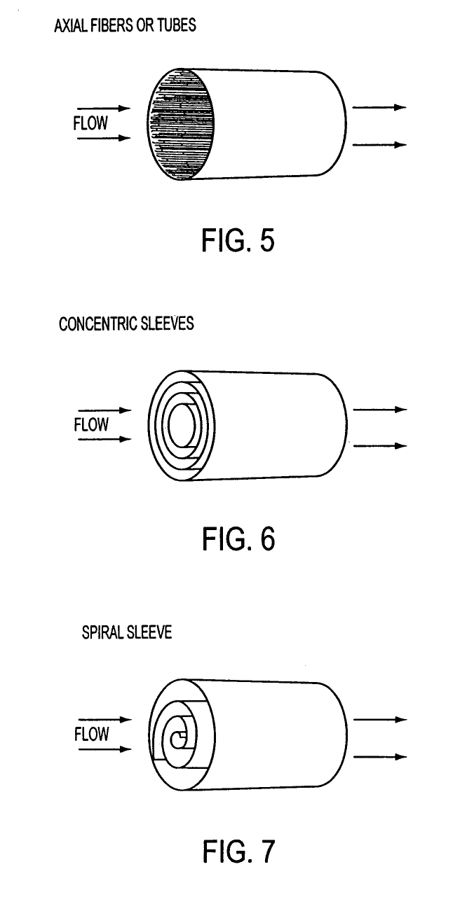

FIG. 5 shows a reactor for our purifying device that contains a plurality of axially aligned tubes or fibers. Preferably, the tubes or fibers are very thin. Optionally, the opposing ends of each tube or fiber can be secured to one of two end plates, not shown in FIG. 5. A direct or axial fluid flow can be passed through the reactor chamber and outside of the axially aligned tubes or fibers, as shown in FIG. 5. A photoreactive component is deposited on the surface of the axially aligned tubes or fibers to form the photoreactive surface. Although not shown in FIG. 5, the activating visible light source in this third embodiment might be placed in any of a number of locations within the reactor. For example, the light source may be placed at the core of the reactor or may be placed along the peripheral wall or walls of the reactor and reflected toward the axially aligned tubes on which the photocatalyst is deposited.

The light source also may be designed to pass through the interior of each of the axially-aligned tubes or fibers made of a translucent material (e.g., plastic or glass). Moreover, such tubes or fibers can be covered with the photoreactive component. However, if this design is adopted and the photoreactive component that is used includes a photocatalyst, such as TiO₂, together with a photosensitizer, as described earlier, then each of these elements of the photoreactive component must be placed onto the outside surface of the axially aligned tubes or fibers. On the one hand, the photocatalyst (e.g., TiO₂) could be placed first onto the surface of the tube or fiber. Thereafter, the photosensitizer could be deposited onto the surface of the photocatalyst. Alternatively, the photosensitizer could be placed first onto the surface of the tube or fiber, and the photocatalyst could be deposited thereon afterwards. However it should be recognized that, even with this sequential application of the photocatalyst and photo sensitizer, a solid layer of photosensitizer over top of a solid layer of the photocatalyst may not be created, but rather a more patchy distribution of these compounds may be created, which nonetheless is acceptable and may even be beneficial for the functioning of our fluid purifying device.

In yet another embodiment of our fluid purifying device, the reactor chamber can contain a series of concentric sleeves, as shown in FIG. 6, that forms the substrate for the photoreactive surface. The sleeves can be made of anything that is sufficiently durable, and preferably flexible, and onto which the photoreactive component can be deposited both on the inner and outer surfaces of each sleeve. For example, the sleeves can be made of paper, plastic or a metallic component, and need not even be entirely continuous but rather may be made of a mesh or other discontinuous structure. However, preferably, a flexible component can be used to facilitate manufacture of the device. An axial fluid flow can be passed through the reactor chamber. As with the other embodiments discussed above, the activating light source could be placed in any convenient location so long as it sufficiently illuminates the photoreactive surfaces to allow for effective decontamination of the fluid stream passing though the fluid purifying device. For example, the light could be placed at the core of the reactor chamber, at either end of the chamber, or even along the inner walls of the reactor chamber.

FIG. 7 shows a reactor for our purifying device that contains a spiral sleeve, i.e., a curved, sheet-like structure, onto which a photoreactive component is preferably deposited onto all available surfaces of the structure to create the photoreactive surface. Although the spiral sleeve could be curved in many different ways, preferably the structure is loosely coiled around itself, much the way one might loosely roll up a piece of paper. Like the series of concentric sleeves utilized in the embodiment of the device provided in FIG. 6, the spiral sleeve can be made of anything that is durable and, preferably, flexible, including paper, plastic or a metallic component, and need not even be entirely continuous but rather may be made of a mesh or other discontinuous structure. The activating visible light source in this fourth alternative reactor embodiment might be placed in any of a number of locations within the reactor. For example, the light source may be placed at the core of the reactor or along the peripheral wall or walls inside the reactor. Moreover, an axial fluid flow, as shown in FIG. 7, can be passed through the reactor chamber.

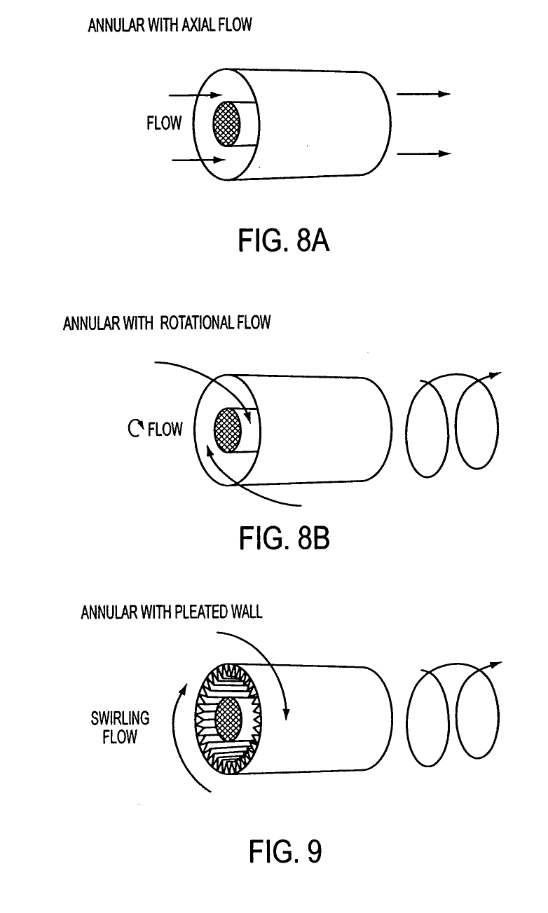

As shown in our preferred embodiment depicted in FIG. 1, when the light source is positioned along the central axis of a cylindrically-shaped fluid purifying device, for example, together with an energy source (e.g., batteries), the internal reactor chamber of the device will take on an annular form. FIGS. 8A and 8B show a more schematic depiction of this annular structure. FIG. 8A shows this annular structure with axial (i.e., direct) fluid flow through the reactor chamber, whereas FIG. 8B shows this annular structure with rotational fluid flow through the reactor chamber. In this simple reactor design, the photoreactive surface might be placed along the inner walls of the reactor chamber.

The surface area of the photoreactive surface in this type of reactor chamber can be increased in a number of ways as described earlier in the context of fluid purifying devices containing a simple cylindrical reactor (i.e., a packed or fluidized bed, an assortment of randomized fibers, a collection of axial fibers, a series of concentric sleeves, or a spiral sleeve). However, other structures for the photoreactive surface also could be used as shown in FIGS. 9 to 12 and as described below.

For example, FIG. 9 shows an annular structure for the reactor chamber of our fluid purifying device, in which a pleated wall serves as the photoreactive surface. The pleated wall could be made of any suitable and sufficiently durable component, such as paper, plastic or metal. The photoreactive component preferably would be deposited onto the pleated wall on the side (or sides) of the wall that is (are) exposed to the activating light source. Although the activating light source could be provided at the core of the reactor device, it also could be placed at one or both ends of the reactor or along the inner walls of the reactor. While an axial fluid flow could be passed through this type of a reaction chamber, a swirling or rotational fluid flow also could be used, as shown in FIG. 9.

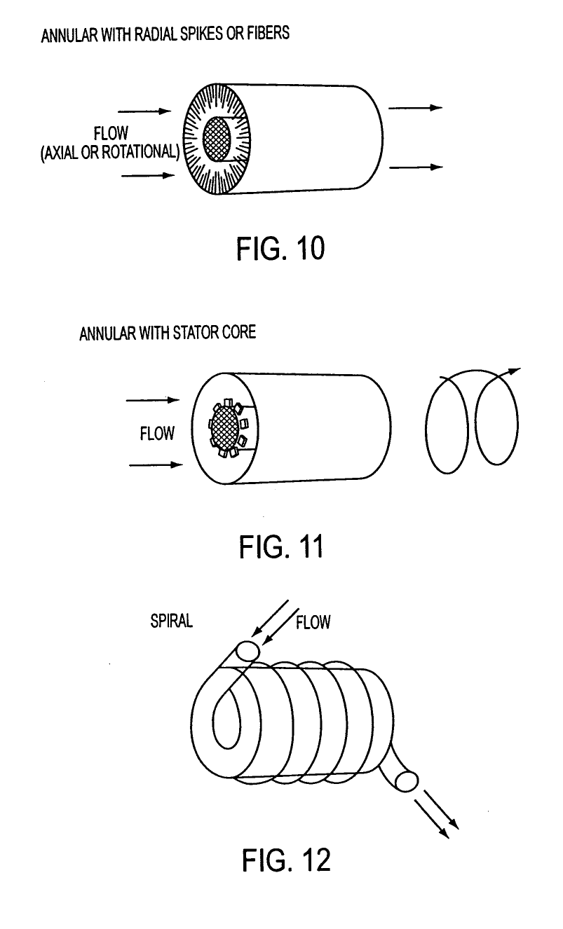

FIG. 10 also shows an annular structure for the reactor chamber of the fluid purifying device, but with an entirely different type of photoreactive surface. The photoreactive surface is akin to a shag carpet preferably placed peripherally, along the inside wall or walls of the reactor, with the radial spikes or fibers of the carpet-like structure facing in towards the core of the reactor. A suitable photoreactive component is deposited on the radial spikes or fibers of the carpet-like structure to create the photoreactive surface. The radial spikes or fibers can, alternatively, be flexible or rigid, and can be made of any suitable component. The fluid flow can be axial, as shown in FIG. 10, and the light source preferably would be at the core of the reactor or at either end of the reactor.

The spikes or fibers in FIG. 10 could be made, for example, of activated carbon, such that the spikes/fibers would have a dual function. On the one hand, they would serve as the substrate for the photoreactive component. On the other hand, they would be capable of absorbing some of the contaminants in the fluid stream (e.g., malodors) for a period of time, and then release the contaminants slowly to then be degraded at the photoreactive surface. That is, the use of this activated carbon would help regulate the doses of contaminant present at the photoreactive surface to allow for more steady, efficient reduction of contaminants in the fluid stream. By absorbing excess amounts of contaminant in the fluid stream and then releasing such excess slowly for immediate degradation by the photocatalyst, a consumer would find the device to be more effective in reducing contaminants. The use of activated carbon in this embodiment of our fluid purifying device is much different than in the conventional situation. As noted previously, conventional devices often use a packed bed of activated carbon that creates back pressure limitations on the decontamination efficiency of such conventional devices since air cannot move freely through the device. In the discussed embodiment, only spikes of activated carbon are proposed for use that would not significantly impede air flow through the reactor chamber.

In yet another embodiment, FIG. 11 shows an annular structure for the reactor of our fluid purifying device. A plurality of fan blades (i.e., the stator) is disposed at the core of the reactor. In operation, the rotating stator imparts a rotational flow to the fluid passing through the device, even when the incoming flow is axial. The photoreactive surface can be formed along the inner walls of the reactor chamber. Alternatively, the surfaces of the fan blades of the stator may be coated with a photoreactive component. The activating visible light source in this alternative reactor embodiment might be placed in any of a number of locations within the reactor. For example, the light source may be placed at the core of the reactor or along the peripheral wall or walls of the reactor.

In yet another embodiment of our fluid purifying device, the reactor of our purifying device contains a coiled or spiraling tube made, for example, of glass, depicted in FIG. 12, through which a fluid containing contaminants to be removed is capable of flowing. In this embodiment, the light source, for example, could be provided outside of the spiraling tube in the annular space of the spiral. Moreover, a photoreactive component could be deposited along the inner walls of the coiled tube. If this is the case, the photocatalyst could be deposited first, and the photosensitizer would then be deposited thereafter. Alternatively, the photosensitizer could be deposited first, followed by the photocatalyst. As noted in the context of our discussion of the reactor design in FIG. 5, the photocatalyst and photosensitizer may have a patchy appearance even after such sequential application, which is acceptable in our device.

Instead of having to rely primarily in this reactor design on diffusion to move a contaminant molecule in a fluid to the photoreactive surface for decomposition (as is the case in many of the previously-described reactor designs in this application), this embodiment also benefits from the turbulent flow of the fluid through the coiled tube to more rapidly move a contaminant molecule to the photoreactive surface for decomposition. Consequently, we envision that this alternative reactor embodiment of our purifying device may be capable of purifying a fluid particularly efficiently. A fan used to direct the flow of fluid through a cylindrical reactor, as might be used in some of the previously-described reactor designs, can initially impose turbulent flow to the fluid. This turbulent flow provides a perpendicular velocity component to the movement of the fluid (and the contaminant molecules within it) that leads to a more rapid flow of contaminant molecules toward the photoreactive surface. However, this flow will have a tendency to straighten out and become more axial or direct such that the rate of decontamination may decrease. In contrast, in a coiled tube, the flow will remain turbulent throughout its flow in the tube.

The following Examples are merely illustrative of the present invention and are not to be considered as limiting the invention, which is properly delineated in the following claims. Unless otherwise noted, the examples refer to the testing of our inventive concept and fluid purifying device to reduce airborne contaminants.

### EXAMPLE 1

### Preparation of Photosensitized Titanium Dioxide

This example describes the preparation of photosensitized titanium dioxide using various ruthenium-based photosensitizers. The procedure utilized here generally follows the procedure set forth in U.S. Pat. No. 6,245,988 B1 to Grätzel, et al., referred to previously and incorporated herein by reference in its entirety (see, for e.g., column 13, line 40 to column 14, line 47), with a few variations as described below.

### A. Photosensitizer is cis-bis(isocyanato) (2,2′bipyridyl-4,4′dicarboxylato)ruthenium (II)

Aggregated particles of titanium dioxide available commercially (e.g., Nanotek TL-1501-000-025, supplied by Nanophase, Burr Ridge, Ill.) were broken down into separate particles, as described in the Grätzel, et al., patent. Thereafter, surface moisture on titanium dioxide was removed. One gram of titanium dioxide was heated to 500 degrees Centigrade for 30 minutes. The resulting dry powder was allowed to cool under argon. It was then transferred immediately to 100 grams of a 2×10⁻⁵ molar solution of cis-bis(isocyanato) (2,2′bipyridyl-4,4′-dicarboxylato) ruthenium (II) (Ruthenium 505, supplied by Solaronix, Aubonne, Switzerland) in ethanol. The slurry was stirred for 12 hours. The resulting coated titanium dioxide was collected by filtration on a 90 cm number 42 Whatman filter paper, and then dried under vacuum.

Although we generally followed the Grätzel, et al. method, we made some changes because we found that the method used to process the photosensitized titanium dioxide impacts the percent reduction that we can get with our fluid purifying device. For example, we found that the acid stabilizer that we used was better than the acetylacetone used in the Grätzel, et al., method, since the latter poisoned our photoreactive surface, thus rendering our fluid purifying device inoperable. Moreover, we found that a 2×10⁻⁵ M solution of the ruthenium complex in ethanol led to greater contaminant reductions in our device than using a 2×10⁻⁴ M solution, as suggested in the Grätzel, et al. method. Also, when we coated titanium dioxide that had not previously been dried, our device reduced contaminants to a lesser extent. By using the procedure described above, we were capable of reducing airborne contaminants entirely (or more accurately stated, to the limits of our detection).

### B. Photosensitizer is cis-bis(isothiocyanato)bis(2,2′-bipyridyl-4,4′-dicarboxylato)-ruthenium (II)

The same procedure described in A was used. Cis-bis(isothiocyanato)bis(2,2′-bipyridyl-4,4′-dicarboxylato)-ruthenium (II) was also supplied by Solaronix, Aubonne, Switzerland under the tradename Ruthenium 535.

### C. Photosensitizer is tris(isothiocyanato)-ruthenium (II)-2,2′:6′2″-terpyridine-4,4′,4″-tricarboxylic acid

The same procedure described in A was used. Tris(isothiocyanato)-ruthenium(II)-2,2′:6,2″-terpyridine-4,4′,4″-tricarboxylic acid was also supplied by Solaronix, Aubonne, Switzerland under the tradename Ruthenium 620 “Black Dye.”

### EXAMPLE 2

### Odor Removal Experiment

A. Control (Ultraviolet Light Plus Titanium Dioxide)

Gas mixtures of a tracer molecule (i.e., isopropyl alcohol) in purified air were generated using a Kin-Tek 485 precision gas standards generator. The generator was set to deliver 8 parts per million of the molecule in purified air. The gaseous mixture was passed into a chamber (R J Harvey, Hillsdale, N.J.) and through a 9 cm number 42 Whatman filter paper containing titanium dioxide (1 gram, Nanotek TL-1501-000-025, supplied by Nanophase, Burr Ridge, Ill.). The chamber was cylindrical, with a 3.5 inch diameter and 2 inch length (i.e., a volume of about 19 sq. inches) and a flow rate through the chamber of 32,000 nL, min. The gas stream exiting the chamber was analyzed by gas chromatography.

The photocatalytic reaction was driven by a 500 W Xenon arc lamp. The infrared component (heat) was removed from the light beam using a water filter (ThermoOriel, part# 61945) and the wavelength range and beam intensity were further controlled using appropriate optical filters. For this control experiment, which involved ultraviolet light and titanium dioxide as a photoreactive surface, the incident UV light was limited to wavelengths greater than 280 nm by the borosilicate window of the reaction vessel. A calibrated photodiode (International Light, IL-1400A (meter), SEL033 (visible detector), SSL001A (uv detector) was used to measure the intensity of the incident beam and a thermocouple (Omega Engineering HH23 (meter), 5SC-TT-T-30-36 (probe)) was used to monitor the temperature inside the reaction chamber.

B. Photosensitized Titanium Dioxide Plus Visible Light

The procedure described above in Example 2.A. was used. The titanium dioxide-coated filter paper was replaced with the coated filter papers from procedures 1.A-C. A 435 nm Long-pass filter (Esco Products, S244435) was inserted in the light path to limit the incident light to wavelengths greater than 400 nm.

C. Measurement of Odor Molecule Depletion

As mentioned in Example 2.A. above, gas chromatography was used to analyze the gas stream exiting the reaction chamber. A gas sampling valve equipped with a 10 mL sample loop (Valco Instruments) allowed intermittent sampling of the gas stream. The malodor and any oxidative products of the photocatalytic reaction were separated on a 30-meter DB-WAXETR column (supplied by J. W. Scientific) using a helium carrier. The gas chromatograph (Hewlett-Packard, HP5890) was equipped with an FID for detection of the separated components.

Quantitation of the FID signal response for the malodor was based on the use of a calibrated permeation source in the Kin-Tek 485. The emission rate of the molecule source is calibrated gravimetrically for a given temperature such that the molecule concentration in the gas mixture is set by the purified air (diluent gas) flow rate.

D. Results

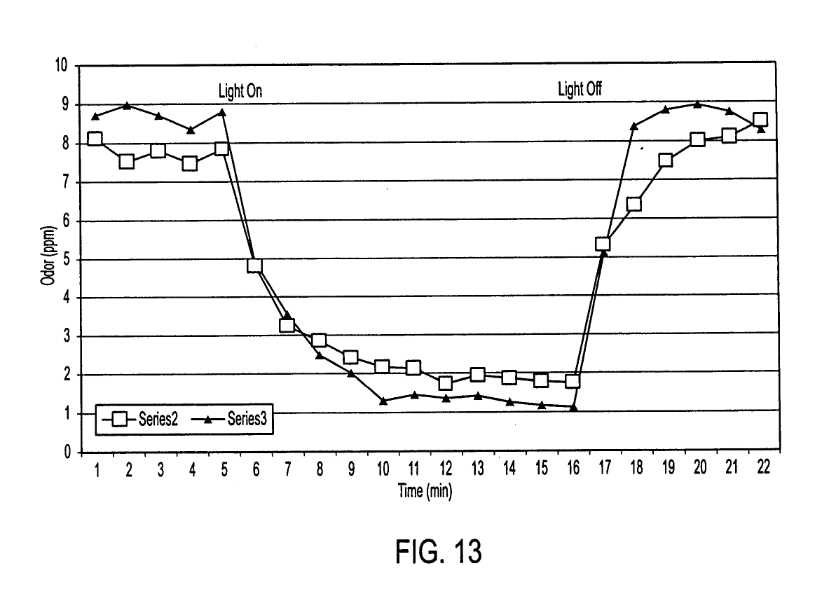

The graph provided at FIG. 13 shows the disappearance of model odor molecules (i.e., isopropyl alcohol) over time in the presence of light. A steady stream of 8 parts per million isopropyl alcohol was flowed through the odor eliminating test chamber. The appropriately filtered light source is switched on. After a brief period of time, the light is switched off.

The line in FIG. 13 with triangle tick points shows the fate of isopropyl alcohol in the test chamber containing simply titanium dioxide in the presence of UV light. When ultraviolet light is switched on, isopropanol disappears below the limits of detection. Our instrument's limit of detection is about one part per million. The line with square tick marks shows the fate of isopropanol in the test chamber containing titanium dioxide coated with Cis-bis(isothiocyanato)bis(2,2′-bipyridyl-4,4′-dicarboxylato)-ruthenium (II) (Example 1.B.) in the presence of visible light. When visible light is turned on, isopropanol disappears. Raw data used to generate the graph provided at FIG. 13 is shown in the following table:

TABLE 1

| Time | Invention | | Control |
|---|---|---|---|
| (sec) | (ppm) | Light | (ppm) |
| 0 | 8.12 | | 8.71 |
| 20 | 7.52 | | 8.98 |
| 30 | 7.8 | | 8.71 |
| 40 | 7.47 | | 8.35 |
| 50 | 7.84 | Light On | 8.8 |
| 60 | 4.81 | | 4.91 |
| 70 | 3.24 | | 3.55 |
| 80 | 2.85 | | 2.48 |
| 90 | 2.42 | | 2.01 |
| 100 | 2.17 | | 1.3 |
| 110 | 2.12 | | 1.45 |
| 120 | 1.73 | | 1.36 |
| 130 | 1.94 | | 1.42 |
| 140 | 1.86 | | 1.26 |
| 150 | 1.78 | | 1.17 |
| 160 | 1.75 | Light Off | 1.12 |
| 170 | 5.32 | | 5.12 |
| 180 | 5.39 | | 8.38 |
| 190 | 5.48 | | 8.8 |
| 200 | 5.68 | | 8.93 |
| 210 | 8.00 | | 8.75 |
| 220 | 8.51 | | 8.28 |

### EXAMPLE 3

### Light Intensity Requirements for our Fluid Purifying Device

This example provides experimental results showing that a fluid purifying device using visible light to excite photosensitized titanium dioxide requires approximately two orders of magnitude greater light intensity compared to that required by one using ultraviolet light and titanium dioxide.

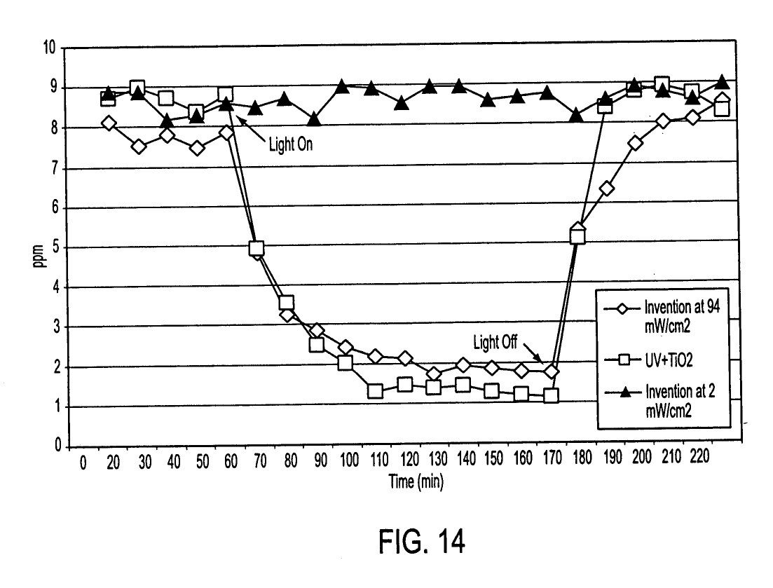

In this example, three experiments were performed. The three experiments used materials described previously in Examples 1 and 2. The model contaminant molecule sought to be removed in this experiment was isopropyl alcohol. Two of the experiments utilized visible light and titanium dioxide coated with photosensitizer (Ru-535; see experimental procedure for preparation of this photoreactive surface in Example 1). The other experiment utilized ultraviolet light and bare titanium dioxide. The two visible light experiments were conducted at two total light intensities, namely between about 1-2 mW/cm² and at about 94 mW/cm². The ultraviolet experiment was conducted at a light intensity of between about 1-2 mW/cm². Total light intensity was measured at the photosensitized titanium dioxide surface using a radiometer or photometer manufactured by International Illumination (model IL 1400A. Results are shown in the graph provided as FIG. 14. The raw data plotted on the FIG. 14 graph is provided in Table 2.

FIG. 14 clearly shows that a higher light intensity at the surface is required for our fluid purifying device which operates upon exposure to a visible light source. This is so even though the photosensitizer employed in this study possesses an incident photon to current efficiency (IPCE) greater than 80% (as reported in M. K. Nazeeruddin et. al., J. Am. Chem. Soc., Vol. 115, pp. 6382-6390, 1993). Consequently, the higher light intensity requirements for our purifying device, which operates upon exposure to visible light, is contrary to that which is expected in view of conventional knowledge as seen, for example, in U.S. Pat. No. 5,790,934 to Says et al. and the Nazeeruddin, et al. article, both discussed earlier.

TABLE 2

| | Invention at | | | Invention at 2 |
|---|---|---|---|---|
| | 94 mW/cm² | | UV + TiO₂ | mW/cm² |
| Time | (ppm) | Light | (ppm) | (ppm) |
| 0 | 8.12 | | 8.71 | 8.88 |
| 20 | 7.52 | | 8.98 | 8.86 |
| 30 | 7.8 | | 8.71 | 8.17 |
| 40 | 7.47 | | 8.35 | 8.28 |
| 50 | 7.84 | Light On | 8.8 | 8.58 |
| 60 | 4.81 | | 4.91 | 8.48 |
| 70 | 3.24 | | 3.55 | 8.69 |
| 80 | 2.85 | | 2.48 | 8.16 |
| 90 | 2.42 | | 2.01 | 8.97 |
| 100 | 2.17 | | 1.3 | 8.93 |
| 110 | 2.12 | | 1.45 | 8.56 |
| 120 | 1.73 | | 1.36 | 8.94 |
| 130 | 1.94 | | 1.42 | 8.94 |
| 140 | 1.86 | | 1.26 | 8.59 |
| 150 | 1.78 | | 1.17 | 8.68 |
| 160 | 1.75 | Light Off | 1.12 | 8.76 |
| 170 | 5.32 | | 5.12 | 8.2 |
| 180 | 6.34 | | 8.38 | 8.59 |
| 190 | 7.48 | | 8.8 | 8.92 |
| 200 | 8 | | 8.93 | 8.78 |
| 210 | 8.1 | | 8.75 | 8.59 |
| 220 | 8.51 | | 8.28 | 8.98 |

### INDUSTRIAL APPLICABILITY

The present invention provides a fluid purifying device. In particular, this device is useful as an air purifying device that is capable of purifying air in the presence of visible light. We envision that the device and related method preferably can be used by consumers in enclosed areas requiring air cleaning, such as, for example, in a bathroom or kitchen. However, we also envision that larger models of the device could be used to purifying fluid streams in larger industrial settings.

While particular embodiments of our invention have been illustrated and described, it will be apparent to those skilled in the art that various changes and modifications may be made without departing from the spirit and scope of the invention. Furthermore, it is intended that the claims will cover all such modifications that are within the scope of the invention.

We claim:

1. A fluid purifying device comprising:
a chamber through which a fluid stream can flow;
a photoreactive surface in the chamber comprising a photocatalyst and a photosensitizer, the photosensitizer having a local maximum absorbance in the visible light range at a wavelength (λₘₐₓ), the photoreactive surface being activatable by visible light having an intensity greater than about 10 mW/cm² in the wavelength band of λₘₐₓ±12 nm; and
a visible light source in the chamber that delivers visible light to the photoreactive surface at an intensity greater than about 10 mW/cm² in the wavelength band of λₘₐₓ±12 nm,
wherein the activated photoreactive surface reduces or eliminates contaminants in the fluid stream when the fluid stream flow through the chamber.

2. The fluid purifying device of claim 1, wherein the fluid stream comprises either water or air.

3. The fluid purifying device of claim 1, wherein the photocatalyst is titanium dioxide.

4. The fluid purifying device of claim 1, wherein the photosensitizer is selected from the group consisting of: (1) blackberry extract and (2) a photosensitizer complex of formula (Ia) MX₃Lₜ or (Ib) MXYLₜ, in which: M is a transition metal preferably such as ruthenium and osmium, but also such as iron, rhenium and technetium; each X is a co-ligand that can be, for example, preferably NCS—, but also Cl^{—}, Br⁻, I⁻, CN⁻, NCO⁻, H₂O, NCH²⁻ and pyridine unsubstituted or substituted by at least one group selected from vinyl, primary, secondary or tertiary amine, OH and C₁₋₃₀ alkyl; Y is a co-ligand that can be o-phenanthroline, or a 2,2′-bipyridine that is unsubstituted or substituted by at least one C₁₋₃₀ alkyl; and Lₜ is a tridentate ligand containing heterocycles such as pyridine, thiophere, imidazole, pyrazole or triazole, carrying at least one carboxylic, phosphoric or hydoxamic acid or a chelating group.

5. The fluid purifying device of claim 1, wherein the photocatalyst titanium dioxide and the photosensitizer is selected from the group consisting of blackberry extract, cis-bis(isocyanato)(2,2′bipyridyl-4,4′-dicarboxylato) ruthenium (II), Cis-bis(isothiocyanato)bis(2,2′-bipyridyl-4,4′-dicarboxylato)-ruthenium (II), and Tris(isothiocyanato)-ruthenium(II)-2,2′:6,2″-terpyridine-4,4′,4″-tricarboxylic acid.

6. The fluid purifying device of claim 1, wherein the contaminants in the fluid stream that are reduced or eliminated are malodors, volatile organic compounds (VOCs), pathogens, mold and bacteria.

7. The fluid purifying device of claim 1, wherein the device additionally comprises:
a fan to direct the flow of the fluid stream through the chamber; and
a power source to operate the fan.

8. The fluid purifying device of claim 7, wherein the power source comprises at least one of (i) an electrical outlet and (ii) at least one battery.

9. The fluid purifying device of claim 7, wherein the device additionally comprises an on/off switch to control the operation of the device.

10. The fluid purifying device of claim 1, wherein the light source comprises at least one light emitting diode.

11. The fluid purifying device of claim 1, wherein the intensity of light emitted from the light source is between about 90 mW/cm² and 1 10 mW/cm².

12. The fluid purifying device of claim 1, wherein the intensity of light emitted from the light source is about 100 mW/cm².

13. The fluid purifying device of claim 1, wherein the device satisfies the requirements of:
$$L \leq V ( \frac{{R}^{2}}{k} ) ( \frac{{μ}^{0.26}}{{ρ}^{0.26} {D}^{1.33}} ) ln ( \frac{{c}_{i n}}{{c}_{out}} ) ,$$
where L is the length of reactor to bring the malodor concentration to zero (cm), V is the velocity of pollutant stream including air or water (cm/s), R is the length scale or dimension for the cross section through which air flows (cm), k is a geometric constant equal to about 0.1 (dimensionless), g is the viscosity of the pollutant stream whether air or water (g/cm*s), ρ is density of air or water depending on the system (g/cm³), D is the diffusion coefficient of malodor in the air or water depending on the system (cm²/s); and cᵢₙ and cₒᵤₜ are the concentrations of malodor in the incoming and outgoing pollutant streams, respectively, whether it be air or water (g/cm³).

14. A method of purifying air in a room comprising:
a step of operating the device of claim 1.

15. A fluid purifying device comprising:
a chamber through which a fluid stream can flow;
a photoreactive surface in the chamber that is activated by visible light having an intensity of at least about 90 mW/cm²; and
a visible light source in the chamber that delivers a light intensity to the photoreactive surface of at least about 90 mW/cm²,
wherein the activated photoreactive surface reduces or eliminates contaminants in the fluid stream when the fluid stream flows through the chamber.

16. The fluid purifying device of claim 15, wherein the fluid stream comprises either water or air.

17. The fluid purifying device of claim 15, wherein the photoreactive surface comprises a photocatalyst and a photosensitizer.

18. The fluid purifying device of claim 17, wherein the photocatalyst is titanium dioxide.

19. The fluid purifying device of claim 17, wherein the photosensitizer is selected from the group consisting of: (1) blackberry extract and (2) a photosensitizer complex of formula (Ia) MX₃Lₜ or (Ib) MXYLₜ, in which: M is a transition metal preferably such as ruthenium and osmium, but also such as iron, rhenium and technetium; each X is a co-higand that can be, for example, preferably NCS—, but also Cl⁻, Br⁻, I⁻, CN⁻, NCO⁻, H₂O, NCH²⁻ and pyridine unsubstituted or substituted by at least one group selected from vinyl, primary, secondary or tertiary amine, OH and C₁₋₃₀ alkyl; Y is a co-ligand that can be o-phenanthroline, or a 2,2′-bipyridine that is unsubstituted or substituted by at least one C₁₋₃₀ alkyl; and Lₜ is a tridentate ligand containing heterocycles such as pyridine, thiophere, imidazole, pyrazole or triazole, carrying at least one carboxylic, phosphoric or hydoxamic acid or a chelating group.

20. The fluid purifying device of claim 17, wherein the photocatalyst titanium dioxide and the photosensitizer is selected from the group consisting of blackberry extract, cis-bis(isocyanato)(2,2′bipyridyl-4,4′-dicarboxylato)ruthenium (II), Cis-bis(isothiocyanato)bis(2,2′-bipyridyl-4,4′-dicarboxylato)-ruthenium (II), and Tris(isothiocyanato)-ruthenium(II)-2,2′:6,2″-terpyridine-4,4′,4″-tricarboxylic acid.

21. The fluid purifying device of claim 15, wherein the photoreactive surface comprises a Ti—O—N constitution in which nitrogen (N) is incorporated in a titanium oxide crystal.

22. The fluid purifying device of claim 15, wherein the contaminants in the fluid stream that are reduced or eliminated are malodors, volatile organic compounds (VOCs), pathogens, mold and bacteria.

23. The fluid purifying device of claim 15, wherein the device additionally comprises:
a fan to direct the flow of the fluid stream through the chamber; and
a power source to operate the fan.

24. The fluid purifying device of claim 23, wherein the power source comprises at least one of (i) an electrical outlet and (ii) at least one battery.

25. The fluid purifying device of claim 23, wherein the device additionally comprises an on/off switch to control the operation of the device.

26. The fluid purifying device of claim 23, wherein the light source comprises one or more light emitting diodes.

27. The fluid purifying device of claim 15, wherein the light intensity is between about 90 mW/cm² and 110 mW/cm².

28. The fluid purifying device of claim 15, wherein the light intensity is about 100 mW/cm².

29. The fluid purifying device of claim 15, wherein the device satisfies the requirements of:
$$L \leq V ( \frac{{R}^{2}}{k} ) ( \frac{{μ}^{0.26}}{{ρ}^{0.26} {D}^{1.33}} ) ln ( \frac{{c}_{i n}}{{c}_{out}} ) ,$$
where L is the length of reactor to bring the malodor concentration to zero (cm), V is the velocity of pollutant stream including air or water (cm/s), R is the length scale or dimension for the cross section through which air flows (cm), k is a geometric constant equal to about 0.1 (dimensionless), F is the viscosity of the pollutant stream whether air or water (g/cm*s), ρ is density of air or water depending on the system (g/cm³), D is the diffusion coefficient of malodor in the air or water depending on the system (cm²/s); and cᵢₙ and cₒᵤₜ are the concentrations of malodor in the incoming and outgoing pollutant streams, respectively, whether it be air or water (g/cm³).

30. A method of purifying the air in a room comprising:
a step of operating the device of claim 15.

31. A fluid purifying device comprising:
a chamber through which a fluid stream can flow;
a photoreactive surface in the chamber that is activated by visible light and that comprises a Ti—O—N constitution in which nitrogen (N) is incorporated in titanium oxide crystal; and
a visible light source in the chamber,
wherein the activated photoreactive surface reduces or eliminates contaminants in the fluid stream when the fluid stream flows through the chamber.

32. The fluid purifying device of claim 31, wherein the light intensity is between about 90 mW/cm² and 110 mW/cm².

33. The fluid purifying device of claim 31, wherein the light intensity is about 100 mW/cm².

34. The fluid purifying device of claim 31, wherein the fluid stream comprises water or air.

35. The fluid purifying device of claim 31, wherein the contaminants in the fluid stream that are reduced or eliminated are malodors, volatile organic compounds (VOCs), pathogens, mold and bacteria.

36. The fluid purifying device of claim 31, wherein the device additionally comprises:
a fan to direct the flow of the fluid stream through the chamber; and
a power source to operate the fan.

37. The fluid purifying device of claim 36, wherein the power source comprises at least one of (i) an electrical outlet and (ii) at least one battery.

38. The fluid purifying device of claim 36, wherein the device additionally comprises an on/off switch to control the operation of the device.

39. The fluid purifying device of claim 31, wherein the light source comprises one or more light emitting diodes.

40. The fluid purifying device of claim 32, wherein the device satisfies the requirements of:
$$L \leq V ( \frac{{R}^{2}}{k} ) ( \frac{{μ}^{0.26}}{{ρ}^{0.26} {D}^{1.33}} ) ln ( \frac{{c}_{i n}}{{c}_{out}} ) ,$$
where L is the length of reactor to bring the malodor concentration to zero (cm), V is the velocity of pollutant stream including air or water (cm/s), R is the length scale or dimension for the cross section through which air flows (cm), k is a geometric constant equal to about 0.1 (dimensionless), [t is the viscosity of the pollutant stream whether air or water (g/cm*s), ρ is density of air or water depending on the system (g/cm³), D is the diffusion coefficient of malodor in the air or water depending on the system (cm²/s); and cᵢₙ, and cₒᵤₜ, are the concentrations of malodor in the incoming and outgoing pollutant streams, respectively, whether it be air or water (g/cm³).

41. A method of purifying air in a room comprising:
a step of operating the device of claim 31.
